(19) United States
(12) Patent Application Publication
(10) Pub. No.: US 2004/0141955 A1
(43) Pub. Date: Jul. 22, 2004

(54) Compositions related to a novel endophytic fungi and methods of use

(76) Inventors: Gary A. Strobel, Bozeman, MT (US); Denise Carol Manker, Davis, CA (US); Julien Mercier, Berkeley, CA (US); Jorge Jimenez, Sacramento, CA (US); John Lin, Sacramento, CA (US); Jonathan Thurston, Sacramento, CA (US); Barry Kersting, Davis, CA (US)

Correspondence Address: Michelle L. Samonek, McDonough Holland & Allen PC, 9th Floor, 555 Capitol Mall, Sacramento, CA, 95814, US

(21) Appl. No.: 10/623,432

(22) Filed: Jul. 17, 2003

Related U.S. Application Data
(63) Continuation-in-part of application No. 10/408,209, filed on Apr. 4, 2003.
Application No. 10/408,209 is a continuation-in-part of application No. 10/121,740, filed on Apr. 11, 2002.
(60) Provisional application No. 60/283,902, filed on Apr. 16, 2001.
(60) Provisional application No. 60/363,072, filed on Mar. 11, 2002.

(51) Int. Cl. A01N 63/00; A01N 65/00; A01N 63/04

(52) U.S. Cl. 424/93.5; 424/93.1; 424/400

(57) ABSTRACT

This invention provides pesticidally effective compositions related to a novel endophytic fungi named Muscodor. Specifically, the invention relates to commercially useful formulations of Muscodor and methods for preparing such formulations. It also provides various synthetic pesticidal mixtures of volatile organic compounds isolatable from Muscodor grown on various substrates. Also provided are methods for inhibiting the growth of organisms, such as microbes, insects, and nematodes by exposing such organisms or the habitats thereof to the above Muscodor-related compositions.

### CROSS-REFERENCE TO RELATED APPLICATIONS

This application claims the benefit under 35 U.S.C. § 120 of U.S. patent application Ser. No. 10/408,209, filed on Apr. 4, 2003, which in turn claims the benefit under 35 U.S.C. §120 of U.S. patent application Ser. No. 10/121,740, filed Apr. 11, 2002, which in turn claims the benefit under 35 U.S.C. § 119(e) of U.S. Provisional Application No. 60/283,902, filed Apr. 16, 2001 and of U.S. Provisional Application No. 60/363,072, filed Mar. 11, 2002. The contents of these applications are hereby incorporated by reference into the present disclosure.

### FIELD OF USE

The present invention relates to the fields of microbiology and pesticides and provides compositions and methods for inhibiting the growth of microbes, insects, and nematodes that adversely affect plants, before and after harvest, and building materials. Specifically, it relates to compositions based on or derived from Muscodor albus and methods of using such compositions as pesticides.

### BACKGROUND OF THE INVENTION

Various publications or patents are referred to in parentheses throughout this application. Each of these publications or patents is incorporated by reference herein. If not given in the text, complete citations to each publication are set forth at the end of the specification, immediately preceding the claims.

It is well known that various microorganisms exhibit biological activity so as to be useful to control plant diseases. Although progress has been made in the field of identifying and developing biological pesticides for controlling various plant diseases of agronomic and horticultural importance, most of the pesticides in use are still synthetic compounds that are classified as carcinogens by the EPA and are toxic to wildlife and other non-target species. For example, methyl bromide is widely used as a soil fumigant and to treat postharvest pests. Due to its high toxicity to humans and animals and its deleterious effect on the atmosphere, the use of methyl bromide will soon be eliminated. Thus, there is a great need to find safer replacements for this and other synthetic pesticides.

### SUMMARY OF THE INVENTION

Applicants have discovered methyl bromide alternatives and methods for their use. Specifically, Applicants have discovered (1) commercially useful formulations of a novel endophytic fungus called Muscodor which produces volatile byproducts that are effective pesticides, and (2) synthetic pesticidal mixtures comprised of one or more of these volatile byproducts.

One aspect of the present invention is a commercially viable, pesticidally effective Muscodor carrier formulation comprising a Muscodor culture adhered to a stable microenvironment that contains micronutrients and stabilizing agents. This formulation is moisture-activated, producing Muscodor volatiles only when exposed to moisture from the surrounding environment. Thus, it is a pesticidal composition that is capable of storage.

In another embodiment the Muscodor carrier formulation is encapsulated. Encapsulation protects the Muscodor culture, carrier, and stabilizing agent from interference by pests, which, for example, might be present in soil applications, while still allowing Muscodor volatiles to escape and to inhibit the growth of microbes, insects, and nematodes.

Another aspect of the present invention is a method for preparing the above Muscodor formulations.

This invention also features various synthetic pesticidal mixtures of volatile organic compounds isolatable from Muscodor grown on various substrates, including rye grain, brown rice grit, and potato dextrose agar.

This invention also encompasses methods for inhibiting the growth of organisms, such as microbes, insects, and nematodes by exposing such organisms or the habitats thereof to individual volatile organic compounds isolatable from a Muscodor culture and/or the Muscodor formulation and synthetic pesticidal mixtures described above. This method has both industrial and agricultural applications. For example, in one embodiment it can be used to treat or prevent toxic mold in building materials and buildings. In another embodiment, it can be used to treat or protect fruits, seeds, plants, and the soil surrounding the plants from infestation by a microbe, insect, or nematode.

### DETAILED DESCRIPTION OF INVENTION

Applicants have isolated and characterized novel fungi named Muscodor and two species thereof, Muscodor albus and Muscodor roseus. Partial genomic sequences for M. albus are provided in SEQ ID NOS.: 1 and 2, and partial genomic sequences for M. roseus are provided in SEQ ID NOS.: 3 and 4. An isolated culture of M. albus has been deposited with the NRRL under Accession No. 30457. An isolated culture of M. roseus has been deposited with the NRRL under Accession No. 30458.

M. albus and M. roseus make volatile byproducts (Muscodor volatiles) that are inhibitory and/or lethal to insects, nematodes, and microbes, including microorganisms that infest building materials and microorganisms that cause disease on plants, seeds, fruit, and in soil. Applicants have also discovered that the components of the Muscodor volatiles, either alone, or in various subcombinations, mimic the pesticidal activity of Muscodor. Thus, the present invention is directed toward stable, commercially useful formulations of Muscodor, synthetic mixtures of one or more of the components of the Muscodor volatiles, and methods of using these compositions as pesticides.

The practice of the present invention employs, unless otherwise indicated, conventional techniques of chemistry, microbiology, cell biology, biochemistry and immunology, which are within the skill of the art. Such techniques are explained fully in the literature. These methods are described in the following publications. See, e.g., Sambrook et al. MOLECULAR CLONING: A LABORATORY MANUAL, 2^{nd} edition (1989); CURRENT PROTOCOLS IN MOLECULAR BIOLOGY (F. M. Ausubel et al. eds. (1987)); the series METHODS IN ENZYMOLOGY (Academic Press, Inc.); PCR: A PRACTICAL APPROACH (M. MacPherson et al. IRL Press at Oxford University Press (1991)); and PCR 2: A PRACTICAL APPROACH (M. J. MacPherson, B. D. Hames and G.R. Taylor eds. (1995)).

Although specific embodiments of the present invention will now be described, it should be understood that such embodiments are examples that are merely illustrative of a small number of the many possible specific embodiments that can represent applications of the principles of the present invention. Various modifications obvious to one skilled in the art to which the present invention pertains are within the spirit, scope and contemplation of the present invention as further defined in the appended claims.

Definitions

The singular form “a,” “an” and “the” include plural references unless the context clearly dictates otherwise. For example, the term “a cell” includes a plurality of cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and agriculturally acceptable carriers. “Consisting of” shall mean excluding more than trace elements of other ingredients and substantial method steps for applying the compositions of this invention. Embodiments defined by each of these transition terms are within the scope of this invention.

As used herein, “biological control” is defined as control of a pathogen or insect by the use of a second organism. For example, bacterial toxins, such as antibiotics, have been used to control pathogens. Such toxins can be isolated and applied directly to the plant or the bacterial species may be administered so it produces the toxin in situ.

The term “fungus” or “fungi” includes a wide variety of nucleated spore-bearing organisms that are devoid of chlorophyll. Examples of fungi include yeasts, molds, mildews, rusts, and mushrooms.

The term “bacteria” includes any prokaryotic organism that does not have a distinct nucleus.

“Pesticidal” means the ability of a substance to increase mortality or inhibit the growth rate of pests. The term pesticidal encompasses the terms antimicrobial, insecticidal, and nematicidal, which are defined below.

“Antimicrobial” means the ability of a substance to increase mortality or inhibit the growth rate of one-celled or filamentous organisms, such as bacteria, fungi, protozoa, slime molds, and blue-green algae. The term antimicrobial encompasses the terms fungicidal and bactericidal, which are defined below.

“Fungicidal” means the ability of a substance to increase mortality or inhibit the growth rate of fungi.

“Insecticidal” means the ability of a substance to increase mortality or inhibit the growth rate of insects or their larvae.

“Bactericidal” means the ability of a substance to increase mortality or inhibit the growth rate of bacteria.

“Nematicidal” means the ability of a substance to increase mortality or inhibit the growth rate of nematodes.

The term “culturing” refers to the propagation of organisms on or in media of various kinds. “Whole broth culture” refers to a liquid culture containing both cells and media. “Supernatant” refers to the liquid broth remaining when cells grown in broth are removed by centrifugation, filtration, sedimentation, or other means well known in the art.

An “effective amount” is an amount sufficient to effect beneficial or desired results. An effective amount can be administered in one or more administrations. In terms of treatment and protection, an “effective amount” is that amount sufficient to ameliorate, stabilize, reverse, slow or delay progression of the target infection or disease states. A “pesticidally effective amount” means an amount sufficient to inhibit the growth of a pest.

“Positive control” means a compound known to have pesticidal activity. “Positive controls” include, but are not limited to commercially available chemical pesticides. The term “negative control” means a compound not known to have pesticidal activity. An example of a negative control is water.

The term “metabolite” or “volatile” refers to any compound, substance or byproduct of a fermentation of a microorganism. Volatiles in most instances evaporate readily at ambient temperature and pressure. “Muscodor volatiles” refer to the gaseous byproducts of a culture of Muscodor. “Volatile organic compound” refers to one of the chemical components of Muscodor volatiles.

The term “mutant” refers to a variant of the parental strain as well as methods for obtaining a mutant or variant in which the desired biological activity is similar to that expressed by the parental strain. The “parent strain” is defined herein as the original Muscodor strains before mutagenesis. Mutants occur in nature without the intervention of man. They also are obtainable by treatment with or by a variety of methods and compositions known to those of skill in the art. For example, parental strains may be treated with a chemical such as N-methyl-N′-nitro-N-nitrosoguanidine, ethylmethanesulfone, or by irradiation using gamma, x-ray, or UV-irradiation, or by other means well known to those practiced in the art.

A “formulation” is intended to mean a combination of active agent and another compound, carrier or composition, inert (for example, a detectable agent or label or liquid carrier) or active, such as an adjuvant. Examples of agricultural carriers are provided below. The fungi can also be formulated as a composition, with a carrier, or, alternatively, with at least one chemical or biological pesticide.

All numerical designations, e.g., pH, temperature, time, concentration, and molecular weight, including ranges, are approximations which may be varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are well known in the art.

In order to achieve good dispersion and adhesion of compositions within the present invention, in one embodiment it is advantageous to formulate the whole broth culture, supernatant and/or volatile with components that aid dispersion and adhesion. Suitable formulations will be known to those skilled in the art (wettable powders, granules and the like, or can be microencapsulated in a suitable medium and the like, liquids such as aqueous flowables and aqueous suspensions, volatile compositions and emulsifiable concentrates. Other suitable formulations will be known to those skilled in the art.

A “variant” is a strain having all the identifying characteristics of the strains of this invention and can be identified as having a genome that hybridizes under conditions of high stringency to the genome of the organism, the partial sequence of which has been deposited in the GenBank depository. “Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. Hybridization reactions can be performed under conditions of different “stringency.” In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

A variant is also defined as a strain having a genomic sequence that is greater than 85%, more preferably greater than 90% or more preferably greater than 95% sequence identity to the genome of M. roseus or M. albus. A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example, those described in current protocols in molecular biology (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following. Internet address: www.ncbi.nlm.nih.gov/cgi-bin/BLAST.

Muscodor Carrier Formulation

Muscodor, grown on various substrates, produces substrate-dependent mixtures of volatile organic compounds that are inhibitory and/or lethal to insects, nematodes, and microbes. Applicants have designed commercially useful formulations of Muscodor in which Muscodor cultures of high cell density are provided with (1) suitable nutrients for production of volatile organic compounds, and (2) a stable microenvironment. These formulations are capable of being stored and of producing volatiles that are effective pesticides.

The present invention is directed to Muscodor carrier formulations, which are commercially viable pesticidal compositions comprising a culture of M. albus or M. roseus, a carrier, and a stabilizing agent, wherein the culture and stabilizing agent are adhered to the carrier. Such formulations are capable of storage for several months and are moisture-activated; i.e., non-metabolizing when dry but capable of producing Muscodor volatiles when contacted with moisture, such as moisture from watering, soil, or greenhouse humidity.

The agriculturally acceptable carrier includes any substrate on which Muscodor will grow after the formulation is exposed to moisture. Suitable carriers contain sources of carbon and nitrogen and other micronutrients to promote Muscodor growth and metabolization. In a preferred embodiment the carriers are grains. The term grain, as used herein, includes whole grain and grain particles, such as grit or powder. Various grains may be used, including grain from corn, rye, barley, rice, wheat, oat bean, soy, and the like. In a particularly preferred embodiment the grain is rye grain, brown rice grit, or barley grain. In another preferred embodiment the carriers are absorptive materials, containing suitable carbon and nitrogen sources. Examples of suitable absorptive materials are clay granules and powders and Biodac (available from Kadant Grantek, Inc. Granger, Ind.). Suitable carbon sources include glucose; suitable nitrogen sources include yeast extract and ammonium sulfate.

The stabilizing agent is a substance capable of maintaining the viability of the Muscodor cells. In a preferred embodiment, the stabilizing agent comprises a carbohydrate, such as sucrose, lactose, or trehalose. In a particularly preferred embodiment the carbohydrate is lactose.

Preferred cultures are those in which high cell density without substantial cell metabolization has been achieved. This is accomplished through the selection of an appropriately balanced culture medium and of suitable fermentation conditions, such as time, temperature, and pH. In a preferred embodiment, the culture is grown in liquid medium containing carbon and nitrogen sources. Suitable carbon sources used in the liquid medium are carbohydrates, preferably glucose, sucrose, and starch. Suitable nitrogen sources include protein-containing materials and nitrogen-containing salts, preferably ammonium salts, yeast extract and malt extract. Suitable fermentation conditions are described below in the “Method of Preparing Muscodor Carrier Formulation” section.

In another embodiment, the Muscodor carrier formulation is encapsulated so as to protect the formulation, for example, from soil-borne organisms, but to allow the Muscodor volatiles to escape. Encapsulation materials are well known to those of skill in the art and include various polymeric matrices. In a preferred embodiment, the encapsulation material is a hydrogel, such as alginate.

In another embodiment, the Muscodor formulations are combined with an effective amount of one or more of a fungicide, an insecticide, a nematicide, an antimicrobial, or a food preservative.

Method of Preparing Muscodor Carrier Formulation

The present invention also embodies a method for producing a Muscodor carrier formulation. The method includes (1) growing a culture of Muscodor, (2) inoculating a carrier with the culture of Muscodor, (3) adding a stabilizing agent to the carrier, and (4) drying the carrier. Suitable carriers, culture media, and stabilizing agents are described above.

In a preferred embodiment, a culture of Muscodor is prepared by inoculating the culture medium with a viable seed culture of Muscodor. The culture is grown, with agitation and aeration, at controlled temperature and pH. The culture media and fermentation conditions are optimized so that the culture used to inoculate the carrier has a high density of cells that are not engaged in substantial metabolization. The preferred temperature is preferably between about 20 to 32° C., more preferably between about 23-27° C., and most preferably 25° C. The preferred pH is about 3 to 7, preferably about 2 to 6, and most preferably about 4. After a high density of cells has been produced, preferably after about 2 to 8 days and more preferably after 7 days of fermentation, the whole fermentation broth is harvested.

The harvested whole broth is used to inoculate the sterilized carriers. The fungus in the carriers is allowed to grow at controlled temperature and moisture content for a sufficient period of time to seed the carriers, preferably for about 1 to 10 days, more preferably for about 3 to 8 days, and most preferably for about 7 days. The preferred controlled temperature is about 20 to 30° C. and more preferably 20 to 25° C. The preferred moisture content is about 20 to 80%, more preferably about 30 to 70%, and most preferably about 65%.

A stabilizing agent, such as lactose, trehalose, or sucrose, is added to the carriers to maintain the viability of the Muscodor cells. In a preferred embodiment, addition of the stabilizing agent and inoculation with the Muscodor culture take place at the same time. In another preferred embodiment, addition of the stabilizing agent follows inoculation and growth of the Muscodor culture.

Finally, the carriers are dried for storage. The Muscodor on the dry Muscodor carriers can be reactivated by moisture, either added externally or from the surrounding environments (e.g., soil and air). Various nutrients that are well known to those of skill in the art can be used along with the added water to enhance the growth and volatile production of the dry Muscodor. After the carrier is rehydrated, the reactivated Muscodor produces volatile organic compounds.

The present invention also encompasses a Muscodor carrier formulation that is encapsulated. Various techniques (Lin, et al., 1991), which have been developed for microbial uses, can be adapted to encapsulate Muscodor carrier formulations or a concentrated fungal mass of Muscodor. The Muscodor carrier formulations, before drying, are encapsulated by various polymeric matrices. Alternatively, a concentrated fungal mass of Muscodor, alone or with nutrients, is encapsulated by a polymeric matrix. The capsules are then dried for storage. Similar to the unencapsulated Muscodor carrier formulation, the encapsulated formulation is reactivated for volatile production by exposure to moisture in the surrounding environment.

Synthetic Pesticidal Mixtures

Applicants have identified the volatile organic compounds that comprise the gaseous byproducts of Muscodor cultures grown on different substrates, such as the Muscodor formulations described above. The volatile organic compounds produced by various Muscodor formulations and by M. albus grown on rye grain and potato dextrose agar (PDA) are set forth in Tables 1, 3, 4, and 6 in the Examples section below. One of skill in the art will appreciate that Muscodor can be grown on a variety of substrates and that the resulting volatile organic compounds are readily identifiable, as described in Example 1 below.

Applicants have discovered that synthetic mixtures of volatile organic compounds that comprise either (1) substantially all components of the gaseous byproducts of M. albus, (2) some subcombination of the gaseous byproducts of M. albus, or (3) one component of the gaseous byproducts of M. albus mimic the pesticidal properties of M. albus. Tables 7-12 show various volatile organic compounds and combinations thereof that inhibit the growth of microorganisms at various concentrations.

Thus, the present invention encompasses various synthetic pesticidal mixtures of some or all of the volatile organic compounds isolatable from an isolated culture of Muscodor. Specific embodiments of mixtures derived from the volatile organic compounds isolatable from a Muscodor formulation in which the carrier is brown rice grit or from a M. albus culture grown on rye seed and/or PDA are described below and in the Examples section. Applicants' experimental results also show that certain mixtures of two or more of the volatile organic compounds cause synergistic inhibition of test organisms. As used herein, a synergistic mixture is a mixture of two or more volatile organic compounds wherein the inhibitory effect that the mixture has on a test organism is greater than the sum of the inhibitory effect of each volatile organic compound of the mixture (used alone) on the test organism. Example 10 sets forth examples of such synergistic compositions and one method for determining synergy.

In one embodiment, the synthetic mixture comprises pesticidally effective amounts of at least two of the following compounds: 2-methyl-1-butanol, isobutyl alcohol, isobutyric acid, 3-methyl-1-butanol, 3-methylbutyl acetate, and ethyl propionate. In a preferred embodiment of this mixture the individual volatile organic compounds, if used in a particular mixture, will have the following effective amounts: isobutyric acid—preferably at least 0.046 μl/ml and more preferably between 0.046 μl/ml and 0.92 μl/ml; 2-methyl-1-butanol—preferably at least 0.11 μl/ml and more preferably between 0.11 μl/ml and 0.92 μl/ml; isobutyl alcohol, ethyl propionate, 3-methyl-1-butanol, and 3-methylbutyl acetate—each preferably at least 0.20 μl/ml and more preferably between 0.20 μl/ml and 0.92 μl/ml. (All concentrations in this section are μl of volatile organic compound per ml air.)

In another embodiment, the synthetic mixture comprises pesticidally effective amounts of at least two of the following compounds: 2-methyl-1-butanol, isobutyl alcohol, methyl isobutyrate, isobutyric acid, 3-methyl-1-butanol, 3-methylbutyl acetate, and ethyl butyrate. In a preferred embodiment of this mixture the individual volatile organic compounds, if used in a particular mixture, will have the following effective amounts: isobutyric acid—preferably at least 0.046 μl/ml and more preferably between 0.046 μl/ml and 0.92 μl/ml; 2-methyl-1-butanol—preferably at least 0.11 μl/ml and more preferably between 0.11 μl/ml and 0.92 μl/ml; isobutyl alcohol, ethyl butyrate, 3-methyl-1-butanol, and 3-methylbutyl acetate—each preferably at least 0.20 μl/ml and more preferably between 0.20 μl/ml and 0.92 μl/ml.

In another embodiment, the synthetic mixture comprises pesticidally effective amounts of at least three of the following compounds: 2-methyl-1-butanol, isobutyl alcohol, methyl isobutyrate, isobutyric acid, 3-methyl-1-butanol, 3-methylbutyl acetate, ethyl propionate, and ethyl butyrate. In a preferred embodiment of this mixture the individual volatile organic compounds, if used in a particular mixture, will have the following effective amounts: isobutyric acid—preferably at least 0.046 μl/ml and more preferably between 0.046 μl/ml and 0.92 μl/ml; 2-methyl-1-butanol—preferably at least 0.11 μl/ml and more preferably between 0.11 μl/ml and 0.92 μl/ml; isobutyl alcohol, ethyl butyrate, ethyl propionate, 3-methyl-1-butanol, and 3-methylbutyl acetate—each preferably at least 0.20 μl/ml and more preferably between 0.20 μl/ml and 0.92 μl/ml.

In another embodiment, the synthetic mixture comprises pesticidally effective amounts of at least two volatile organic compounds isolatable from an isolated culture of Muscodor albus grown on potato dextrose agar. A preferred embodiment of this mixture comprises 3-methylbutyl acetate and propionic acid, 2-methyl, 3-methylbutyl ester.

In yet another embodiment, the synthetic mixture comprises pesticidally effective amounts of at least two volatile organic compounds isolated from an isolated culture of M. albus grown on brown rice grit.

In yet another embodiment, the synthetic mixture comprises pesticidally effective amounts of at least three volatile organic compounds isolated from an isolated culture of M. albus grown on rye grain.

In yet another embodiment, the synthetic mixture comprises pesticidally effective amounts of at least three volatile organic compounds isolated from at least one of an isolated culture of Muscodor albus grown on rye grain, an isolated culture of Muscodor albus grown on brown rice grit, and an isolated culture of Muscodor albus grown on potato dextrose agar.

A preferred embodiment of this mixture comprises pesticidally effective amounts of either 2-methyl-1-butanol or 3-methyl-1-butanol, ethyl butyrate, isobutyl alcohol, phenethyl alcohol, ethyl isobutyrate, and isobutyric acid. In a preferred embodiment, the individual volatile organic compounds of the mixture have the following effective amounts: at least 0.11 μl/ml, more preferably between 0.11 μl/ml and 0.64 μl/ml, and most preferably 0.38 μl/ml ethyl butyrate; preferably at least 0.023 μl/ml, more preferably between 0.023 μl/ml and 0.13 μl/ml, and most preferably 0.080 μl/ml isobutyl alcohol and phenethyl alcohol and isobutyric acid; preferably at least 0.015 μl/ml, more preferably between 0.015 μl/ml and 0.092 μl/ml, and most preferably 0.054 μl/ml ethyl isobutyrate; preferably at least 0.030 μl/ml, more preferably between 0.030 μl/ml and 0.18 μl/ml, and most preferably 0.12 μl/ml 2-methylbutyl acetate; and preferably at least 0.25 μl/ml, more preferably between 0.25 μl/ml and 1.48 μl/ml, and most preferably 0.86 μl/ml of either 2-methyl-1-butanol or 3-methyl-1-butanol.

Methods of Using Muscodor Formulations and Synthetic Compositions

As shown in the tables and examples below, Applicants have discovered that the compositions described above—the Muscodor formulations and synthetic pesticidal mixtures—inhibit the growth of, or kill one or more of the following organisms: a microbe, a nematode, and an insect. They are lethal to the major fungal and bacterial pathogens of humans including C. albicans (Table 11) and A. fumigatus and Pseudomonas sp. They kill bacteria that contaminate food such as S. auerus and E. coli (Table 11) and have been found to be lethal to Stachybotrys sp. (contaminator of homes and public buildings) and also a number of wood decay fungi.

Thus, the present invention encompasses methods for inhibiting the growth of an organism selected from the group consisting of microbes, insects, and nematodes by exposing the organism or its habitat to an effective amount of the following Muscodor-derived compositions: (1) a Muscodor carrier formulation, (2) one of the volatile organic compounds isolatable from Muscodor, described in the Examples section below, and (3) mixtures of two or more of the volatile organic compounds isolatable from Muscodor, described above and in the Examples section below. The habitats of the organism will be known to those of skill in the art and include seeds, plants, the soil surrounding plants, farm implements, food, containers of post harvest food, building materials, and the space between building materials.

In preferred embodiments of the invention, inhibition of the growth of microbes, insects, and nematodes is accomplished by exposing the organism or its habitat to an effective amount of 2-methyl-1-butanol, isobutyric acid, 3-methylbutyl acetate, isobutyl alcohol, or 3-methyl-1-butanol. In particularly preferred embodiments, the effective amount of 2-methyl-1-butanol is preferably less than 2500 ppm and the effective amount of isobutyuric acid is less than 2800 ppm.

In a preferred embodiment, the invention provides a method for treating or preventing toxic mold in building materials and buildings by exposing the building, the building materials, or the spaces between the building materials to one or more of the Muscodor-derived compositions described above.

In agricultural applications, the invention provides a method for treating or protecting fruit, seeds, plants, and the soil surrounding the plants, including potting soil mixes, from infestation by a microbe, insect, or nematode by exposing the fruit, seeds, plants, and the soil surrounding the plants to one or more of the Muscodor-derived compositions described above.

### EXAMPLES

### Example 1

### Preparation of a Muscodor Carrier Formulation

A medium (10 liters) at pH of 3.7, which contained yeast extract (5 g/L), glucose (20 g/L), and soluble starch (4 g/L), was sterilized in a fermentor. The fermentor was then inoculated with a viable seed culture (0.2 liter) of Muscodor, and operated at ca. 25° C. The fermentation medium was mechanically agitated (at 300 rpm) and aerated (at 0.3 vvm). After 7-day fermentation, the whole fermentation broth containing a high density of the fungal cells was harvested. This whole broth (0.17 L) was used as inoculum to seed the sterilized brown rice grits (200 g dry grits containing 200 ml of water) in a 2.8-liter flask. The fungus in the carriers was allowed to grow at 20 to 25° C. and a moisture content of ca. 65% for 7 days. 284 ml of lactose solution (10% w/v) was added to the grown Muscodor carriers contained in the flask. The carriers were air dried to a moisture content of 5-15% for storage.

### Example 2

### Identification of Volatile Organic Compounds Produced by a Muscodor Carrier Formulation

Analysis of the volatile components produced by a Muscodor formulation using brown rice grits as a carrier was performed. As a first step, the Muscodor formulation was rehydrated, using 1.78 mL water per gram of M. albus on the carrier. Then, the Muscodor formulation described above (2.5 g) was placed in a 250 mL Erlenmeyer flask and sealed with a rubber stopper. A “Solid Phase Micro Extraction” syringe was used to trap the fungal volatiles. The fiber material (Supelco) was 50/30 divinylbenzene/carburen on polydimethylsiloxane on a stable flex fiber. The syringe was placed through the septum of the rubber stopper and exposed to the vapor phase for 25 min. The syringe was then inserted into a gas chromatograph (Hewlett Packard 5890 Series II) equipped with a flame ionization detector (FID). A 30 m×0.25 mm I.D. ZB Wax capillary column with a film thickness of 0.50 mm was used for the separation of the volatiles. The column was temperature programmed as follows: 31° C. to 220° C. at 5° C./min with a total run time of 43.8 minutes. The injector temperature was 250° C. The carrier gas was Helium Ultra High Purity (local distributor) and the initial column head pressure was 105 kPa. Prior to trapping the volatiles, the fiber was conditioned at 250° C. for 30 minutes under a flow of helium gas. A 30 sec. injection time was used to introduce the sample fiber into the GC. Pure standard compounds were analyzed under the same conditions to confirm the identity of the components of the Muscodor formulation. The volatile organic compound profile observed is shown in Table 1.

TABLE 1

| | M. albus on brown |
|---|---|
| | rice grits (rehydrated) |
| Volatile Organic Compound | Total Area (%) |
| Ethyl isobutyrate | 2.9 |
| (Propanoic acid, 2-methyl, ethyl ester) | |
| Ethyl propionate | 47 |
| (Propanoic acid, ethyl ester) | |
| Isobutyl alcohol | 3.2 |
| (2-methyl-1-propanol) | |
| Isobutyric acid | 5.2 |
| (Propanoic acid, 2-methyl) | |
| Methyl 2-methylbutyrate | 2.6 |
| (Propanoic acid, 2-methyl, methyl ester) | |
| Phenethyl alcohol | 3.6 |
| (Phenylethyl alcohol) | |
| 3-Methylbutyl acetate | 5.5 |
| (1-butanol, 3-methyl, acetate) | |
| 3-Methyl-1-butanol | 28.2 |
| (1-butanol, 3-methyl) | |

### Example 3

### Biological activity of the Muscodor Carrier Formulation in Controlling Damping Off

Samples of the Muscodor formulation were tested over time and at several temperatures for their efficacy in controlling damping off in soil pot tests. Specifically, greenhouse soil mix was infested with Rhizoctonia solani cultures that were grown on PDA for 5-7 days. The cultures from two plates were ground with water in a blender for 30 sec and mixed with one liter of soil (Fafard no. 2). Portions of the R. solani-infested soil were then mixed with one of the following two Muscodor carrier formulations: a carrier containing a sugar stabilizing agent such as lactose (prepared as described in Example 1) or a carrier to which a sugar stabilizing agent had not been added (prepared as described in Example 1, except without the final step of adding lactose before air drying). 100 ml of the Muscodor carrier formulation-treated R. solani-infested soil was placed in each of several plastic pots, with 3-4 replicate pots per treatment. A pathogen-only control as well as a non-infested control were included in each experiment. After an overnight incubation, approximately 70 broccoli seeds were scattered on the surface of each pot and covered with non-infested potting mix, which was then moistened with a spray bottle. At this time the pots were watered by placing them in a tray of water for 1 h, after which the excess water was drained. Water was then added as needed during the course of the experiment. After approximately 6-7 days under fluorescent light, healthy seedlings were counted and results expressed as percentage emergence of the non-infested control. The Muscodor carrier formulations had good efficacy in controlling the damping off disease (caused by R. solani).

As shown in Table 2, the above experiment was conducted with freshly prepared Muscodor carrier formulations (with and without stabilizing agent) and with formulations that had been stored for various time periods, at various temperatures. The Muscodor carrier formulation with the sugar stabilizing agent possessed acceptable stability for commercial application. In contrast, the Muscodor carrier formulations without the sugar stabilizing agent lost pesticidal activity quickly.

TABLE 2
Stability of Dry Brown Rice Grit Carrier
Damping-off assay (R. solani) 2.25 g dry carrier per 300 ml soil
Carrier With Stabilizer

| | Broccoli Seedling Emergence | | | |
|---|---|---|---|---|
| Storage | (% of Non-inoculated Control) | | | |
| Temperature | 0 Days | 1 Month | 3 Month | 6 Month |
| 4 C. | 107.5% | 81.3% | 100.9% | 101.4% |
| Room | 107.5% | 71.3% | 103.7% | 99.2% |
| Temperature | | | | |
| 40 C. | 107.5% | 84.6% | 99.5% | No Test |

Carrier Without Stabilizer

| | Broccoli Seedling Emergence | | | |
|---|---|---|---|---|
| Storage | (% of Non-inoculated Control) | | | |
| Temperature | 0 Days | 1 Month | 2 Month | 4 Month |
| 4 C. | 79.2% | 10.7% | 5.9% | 1.0% |
| Room | 79.2% | 0.0% | 0.0% | No Test |
| Temperature | | | | |
| 40 C. | 79.2% | 8.5% | 0.0% | No Test |

### Example 4

### Preparation of an Encapsulated Muscodor Carrier Formulation

The whole broth of Muscodor prepared via the fermentation process described in Example 1 was centrifuged. The resulting fungal mycelia pellet (10 ml) was added to 90 ml of a 0.3 M CaCl₂ solution containing 5% lactose. This mixture was then added dropwise, using a 60-ml syringe, to a stirred 0.5% (w/v) alginate solution. Approximately 1 liter of sterile deionized water was then added to the capsule-containing alginate solution. The wet capsules were harvested through filtration using filter paper. The capsules were air dried in a biological hood. About 15 dry capsules were added to a 250-ml flask, and 3 ml of potato dextrose broth were then added to the dry capsules. Two days later, some fungal growth was observed and a gaseous sample from the headspace of the flask was analyzed by gas chromatography. As shown in Table 3, typical volatile organic compounds of Muscodor were found in the headspace.

TABLE 3

| | M. albus on |
|---|---|
| | encapsulated material |
| Volatile Organic Compound | Total Area (%) |
| Ethyl butyrate | 0.4 |
| (Butanoic acid, ethyl ester) | |
| Ethyl isobutyrate | 1.2 |
| (Propanoic acid, 2-methyl, ethyl ester) | |
| Ethyl propionate | 41.2 |
| (Propanoic acid, ethyl ester) | |
| Isobutyl alcohol | 3.4 |
| (2-methyl-1-propanol) | |
| Isobutyric acid | 12.6 |
| (Propanoic acid, 2-methyl) | |
| 2-Methylbutyl acetate | 0.34 |
| (1-butanol, 2-methyl, acetate) | |
| 2-Methyl-1-butanol | 35.1 |
| (1-butanol, 2-methyl) | |
| Methyl 2-methyl butyrate | 0.9 |
| (Propanoic acid, 2-methyl, methyl | |
| ester) | |
| Phenethyl alcohol | 1.3 |
| (Phenylethyl alcohol) | |

### Example 5

### Identification of Volatile Organic Compounds Produced by Muscodor Grown on Rye

To produce rye grain culture of M. albus, 150 g of rye grain was placed in a 2 L flask with 250 ml of water and autoclaved twice for 30 minutes on two consecutive days. The flasks were inoculated by adding the content of half of a PDA plate culture cut in small cubes or by pipetting 25 ml of a liquid mycelial suspension. The mycelial suspension was grown by adding small cubes of solid culture to a 1 L flask containing 100 ml of potato dextrose broth and agitated on a rotary shaker. The colonized grain culture was ready to use in 10-15 days.

Analysis of production of volatile organic compounds was carried out as described in Example 2 above. One of the novel components of the gases produced by the Muscodor carrier formulation in Example 2 (ethyl propionate) was produced in high concentration in the rye grain preparation. The volatile organic compound profile observed is shown in Table 4.

TABLE 4

| | M. albus colonized |
|---|---|
| | rye grain |
| Volatile Organic Compound | Total Area (%) |
| Ethyl butyrate | 0.14 |
| (Butanoic acid, ethyl ester) | |
| Ethyl isobutyrate | 0.71 |
| (Propanoic acid, 2-methyl, ethyl | |
| ester) | |
| Ethyl propionate | 9.63 |
| (Propanoic acid, ethyl ester) | |
| Isobutyl alcohol | 1.37 |
| (2-methyl-1-propanol) | |
| Isobutyric acid | 14.9 |
| (Propanoic acid, 2-methyl) | |
| 2-Methylbutyl acetate | 2.4 |
| (1-butanol, 2-methyl, acetate) | |
| 2-Methyl-1-butanol | 48.5 |
| (1-butanol, 2-methyl) | |
| Methyl 2-methylbutyrate | 0.26 |
| (Propanoic acid, 2-methyl, methyl | |
| ester) | |
| Phenethyl alcohol | 5.7 |
| (Phenylethyl alcohol) | |

### Example 6

### Biological Activity of Volatile Organic Compounds Produced by Muscodor Grown on Rye

Activity Against Selected Fungi

The inhibitory and lethal activity of volatiles produced by M. albus on potato dextrose agar (PDA) and rye grain was tested against a number of fungi. For the PDA plate cultures, a moat, free of medium, was cut across each plate to physically separate two agar sections, ensuring that any inhibition was due to air-diffusible compounds only. After growing M. albus for 7 days on one section, three agar plugs of a test fungus were placed on the other section, and the whole plate was sealed with parafilm. After three days, growth of the test fungus were assessed. In the absence of growth, the viability of the test plugs was assessed by transferring them to fresh PDA plates. Plugs that did not show signs of growth after 5 days were considered dead.

Rye grain cultures were tested similarly using a “Y” plate divided in three equal sections with 5 or 10 rye grains in one section and the test plugs placed in another section on PDA. The third section remained empty. Growth and viability of the plugs was assessed as described above.

Results are expressed as growth (G) or no growth (NG) with the number of dead plugs over total plugs in parentheses.

TABLE 5

| Fungus | PDA culture | 5 rye grains | 10 rye grains |
|---|---|---|---|
| Cylindrocarpon sp. strain A | NG (0/6) | NG (3/3) | NG (3/3) |
| Cylindrocarpon sp. strain B | NG (0/6) | NG (3/3) | NG (3/3) |
| Geotrichum candidum | NG (6/6) | NG (3/3) | NG (3/3) |
| Geotrichum citri-auranti | NG (6/6) | NG (3/3) | NG (3/3) |
| Fusarium oxysporum strain A | G | G | NG (2/3) |
| Fusarium oxysporum strain B | NG (0/3) | NG (0/3) | NG (3/3) |
| Rhizoctonia solani | NG (3/3) | NG (3/3) | NG (3/3) |
| Trichoderma sp. | G | G | G |

Although some pathogens, such as G. candidum, G. citri-auranti and R. solani were inhibited and killed regardless of the type of M. albus culture used, rye culture proved more active against harder to kill pathogens such as Cylindrocarpon and F. oxysporum. Trichoderma, a non-pathogenic fungus, proved to be insensitive to M. albus volatiles regardless of the culture or dose used.

Activity Against Beet Armyworm (Spodoptera exigua)

Three small plastic beakers containing approximately 150 grams of autoclaved rye seed colonized with M. albus were placed in a plastic box (approximately 250 in²). A companion box was set up at room temperature without the three beakers of fungus. Both boxes contained a Petri plate of PDA with a small plug of Rhizoctonia solani in the center, as a bioassay indicator. 96-well microtitre plates containing beet armyworm eggs that had been overlaid onto artificial diet were introduced into each box. After two days, the eggs in the box without the Muscodor began to hatch, and the R. solani developed new mycelia. The armyworm eggs did not hatch in the box containing the rye culture of M. albus. Moreover, the growth of R. solani was suppressed. After 5 days, the armyworms in the untreated box had achieved second to third instar.

In another experiment, paired microtitre plates containing armyworm larvae that had been grown for three days on artificial diet were introduced into the boxes. The plate in the Muscodor box ceased feeding and remained stunted compared to the untreated controls. After five days, the armyworms in the treated plate were dead.

Activity Against Corn Rootworm Beetles (Diabrotica undecimpunctata)

Paired microtitre plates with corn rootworm eggs that had been overlaid onto artificial diet were also introduced into the boxes. The eggs had just begun to hatch when the plates were introduced into the test boxes. Approximately half of the eggs hatched in the Muscodor box. The remainder did not hatch, and all of the neonates were dead within two days. The microtitre plate in the untreated control box developed a normal infestation that progressed with 3-6 third-instar grubs per well, after one-week.

### Example 7

### Identification of Volatile Organic Compounds Produced by Muscodor Grown on Potato Dextrose Agar

Cultures of Muscodor albus were grown on potato dextrose agar (PDA) in Petri plates. A method was devised to analyze the gases in the air space above the M. albus mycelium growing in Petri plates. A “Solid Phase Micro Extraction” syringe was used to trap the fungal volatiles. The fiber material (Supelco) was 50/30 divinylbenzene/carburen on polydimethylsiloxane on a stable flex fiber. The syringe was placed through a small hole drilled in the side of the Petri plate and exposed to the vapor phase for 45 min. The syringe was then inserted into a gas chromatograph (Hewlett Packard 5890 Series II Plus) equipped with a mass-selective detector. A 30 m×0.25 mm I.D. ZB Wax capillary column with a film thickness of 0.50 mm was used for the separation of the volatiles. The column was temperature programmed as follows: 25° C. for 2 min followed to 220° C. at 5° C./min. The carrier gas was Helium Ultra High Purity (local distributor) and the initial column head pressure was 50 kPa. The He pressure was ramped with the temperature ramp of the oven to maintain a constant carrier gas flow velocity during the course of the separation. Prior to trapping the volatiles, the fiber was conditioned at 240° C. for 20 minutes under a flow of helium gas. A 30 sec. injection time was used to introduce the sample fiber into the GC. The gas chromatograph was interfaced to a VG 70E-HF double focusing magnetic mass spectrometer operating at a mass resolution of 1500. The MS was scanned at a rate of 0.50 sec. per mass decade over a mass range of 35-360 amu. Data acquisition and data processing was performed on the VG SIOS/OPUS interface and software package. Initial identification of the unknowns produced by M. albus was made through library comparison using the NIST database.

Comparable analyses were conducted on Petri plates containing only PDA and the compounds obtained therefrom, mostly styrene, were subtracted from the analyses done on plates containing the fungus. Final identification of 20 out of 28 compounds was done on a comparative basis to authentic standards using the GC/MS methods described herein. However, 8 other compounds composing only approximately 20% of the volatiles have only been tentatively identified on the basis of the NIST data base information and were not included in any of the bioassay tests that employed artificial mixtures of M. albus compounds.

The volatile organic compound profile observed is shown in Table 6 below. In the table, the symbol * denotes that no molecular-ion peak was observed in the spectrum of either the standard compound or the compound undergoing the analysis. The symbol # denotes that a spectrum and retention time of this component was observed and the substance matched to the most likely compound in the NIST data base, but the data have not been confirmed by use of an appropriate identical standard compound by either retention time or MS. These compounds were not placed in the artificial mixture in the bioassay test.

TABLE 6
GC/MS analysis of the volatile
compounds produced by M. albus.

| | Total | | | |
|---|---|---|---|---|
| RT | Area (%) | M/z | Possible compound | MW |
| 3:45 | 0.33 | 114 | Octane | 114 |
| 4:19 | 0.93 | 58 | Acetone | 58 |
| 4:37 | 0.68 | 74 | Methyl acetate | 74 |
| 5:56 | 7.63 | 88 | Ethyl acetate | 88 |
| 6:51 | 0.31 | 102 | Propanoic acid, 2-methyl, methyl ester | 102 |
| 7:16 | 6.24 | * | Ethanol | 46 |
| 8:03 | 2.07 | 116 | Propanoic acid, 2-methyl-ethyl ester | 116 |
| 11:45 | 0.58 | * | Propanoic acid, 2-methyl 2-methylpropyl ester | 144 |
| 12:05 | 2.06 | 74 | Isobutyl alcohol | 74 |
| 12:50 | 22.24 | * | 1-butanol, 3-methyl, acetate | 130 |
| 14:57 | 1.53 | * | Propanoic acid, 2-methyl, 3-methylbutyl ester | 158 |
| 15:28 | 22.99 | * | 1-butanol, 3-methyl- | 88 |
| 16:08 | 0.29 | 138 | #Furan, 2-pentyl- | 138 |
| 18:53 | 0.29 | 142 | #4-nonanone | 142 |
| 20:38 | 0.41 | 142 | 2-nonanone | 142 |
| 21:07 | 0.30 | 204 | # Naphthalene, | 204 |
| | | | decahydro-4a-methyl-1-methylene-7-(1-methylethylidene)-, | |
| | | | (4aR-trans)- | |
| 22:54 | 1.51 | 204 | # Azulene, | 204 |
| | | | 1,2,3,4,5,6,7,8-octahydro-1,4-dimethyl-7-(1-methylethenyl)-, | |
| | | | [1S-(1.alpha.,4.alpha.,7.alpha.)] | |
| 23:16 | 0.94 | 204 | # Cyclohexene, | 204 |
| | | | 4-(1,5-dimethyl-1,4-hexadienyl)-1-methyl- | |
| 25:20 | 3.63 | 204 | # 1H-3a,7-methanoazulene, | 204 |
| | | | 2,3,4,7,8,8a-hexahydro-3,6,8,8 tetramethyl-, | |
| | | | [3R-(3.alpha., 3a.beta.,7.beta.,8a.alpha.)] | |
| 25:30 | 6.08 | 88 | Propanoic acid, 2-methyl | 88 |
| 26:04 | 0.48 | 204 | Caryophyllene | 204 |
| 27:55 | 0.34 | 204 | # | 204 |
| | | | Naphthalene,1,2,4a,5,6,8a-hexahydro-4,7-dimethyl- | |
| | | | 1-(1-methylethyl)-, [1R-(1.alpha., | |
| | | | 4a.alpha.,8a.alpha.)] | |
| 28:34 | 0.36 | 204 | # | 204 |
| | | | Spiro[5.5]undec-2-ene,3,7,7-trimethyl-11-methylene | |
| 28:50 | 1.07 | 204 | Azulene, 1,2,3,5,6,7,8, 8a-octahydro-1, | 204 |
| | | | 4-dimethyl-7-(1-methylethyenyl)-, | |
| | | | [1S-(1.alpha.,7.alpha.,8a.beta.)] | |
| | | | Common Name: Bulnesene | |
| 28:57 | 3.24 | 204 | Naphthalene, | 204 |
| | | | 1,2,3,5,6,7,8,8a-octahydro-1,8a-dimethyl-7-(1- | |
| | | | methylethenyl)-,[1R-(1.alpha.,7.beta., | |
| | | | 8a.alpha.)] | |
| | | | Common Name: Valencene | |
| 31:12 | 1.74 | * | Acetic acid,2-phenylethyl ester | 164 |
| 33:17 | 1.06 | 122 | Phenylethyl alcohol | 122 |
| 39:00 | 9.76 | 204 | Unknown | 204 |

### Example 8

### Biological Activity of Volatile Organic Compounds Produced by Muscodor Grown on PDA

Fungal and Human Pathogens

A strip of agar was removed from the middle of PDA plates, creating two approximately equal and separate sections where microorganisms could grow, as described by Strobel et al., 2001. One agar plug of M. albus culture was placed on one section and grown for 10 days with the plates enclosed in a plastic bag. After ten days, the other section was inoculated with various fungal pathogens, with sectioned plates without M. albus serving as control. There were three plates for each treatment. Penicillium expansum, Monilinia fructicola, Candida albicans and bacteria were applied as a spore/cell suspension, while the other pathogens were applied as a single 3 or 6 mm mycelial plug in each plate. Pathogen growth, measured by colony diameter, was evaluated after 3 days. Reisolation of pathogens, to evaluate their viability, was attempted at the end of the experiments by lifting the agar in the inoculated area and transferring it to fresh PDA plates.

None of the pathogens, except F. solani and F. oxysporum lycopersici, grew in the presence of M. albus (Table 11) and their growth was inhibited. In addition, the volatiles of M. albus did not kill Xylaria sp., a close relative of M. albus, although they did inhibit the growth of Xylaria sp. (Table 11).

Nematode (Caenorhabditis elegans)

Plates using the moat system (Worapong et al., 2001) were inoculated on one side with M. albus, and on the opposite side with E. coli, or free-living nematodes with E. coli. Identical plates were set up without the Muscodor. After five-days the plate without the Muscodor had developed a large reproducing population of nematodes which crossed the moat and were beginning to populate the opposite side of the Petri dish. The E. coli had grown to normal colony morphology on the companion plate. The Muscodor treated plate had developed a substantial colony that was sending mycelia across the surface of the PDA. The nematodes that were present were sluggish, yet motile. By seven days, the Muscodor reached the edge of the PDA and was sending mycelia into the moat of the plate with E. coli, and the plate with the round worms. Only a small number of living adult nematodes were present on the agar, and their mobility was limited.

### Example 9

### Sourcing of Volatile Organic Compounds Isolatable from Muscodor

The majority of the volatile organic compounds produced by M. albus on the substrates described above were obtained from Aldrich Chem Co., however, valencene was obtained from Fluka Chem Co. and synthetic bulnesene was obtained from Dr. Clayton Heathcock of U.C. Berkeley, Dept of Chemistry and can be synthesized following the procedures of Heathcock and Ratcliffe (1971).

2-methyl butyl isobutyrate and 2-methyl butyl acetate can be synthesized following the procedures below.

2-Methylbutyl isobutyrate (1-Butanol, 2-methyl, Propionate, 2-methyl). To a solution of isobutyric acid (1.05 mL, 11 mmol) in 5 mL of dry CH₂Cl₂ at 0° C. was added oxalyl chloride (5.65 mL, 2.0 M in hexanes) and allowed to stirred for 1 hr. Then slowly added 2-methyl-1-butanol (1.23 mL, 0.011 mol) and allowed to stirred at room temperature for 12 hrs. Reaction was quenched by addition of dilute NaHCO₃ and extracted with hexanes (2×5 mL). The organic layers were combined and the solvent was removed carefully under a stream of air. ¹H NMR (400 MHz, CDCl₃) δ 3.95 (t, J=6.4 Hz, OCH₂CH), 2.37 (m, CH), 1.55 (m, CHₐH_{b}CH₃), 1.37 (m, CH(CH₃)CHₐCH_{b}), 1.01 (d, J=7.2 Hz, (CH₃)₂CH), 0.77 (d, J=6.4 Hz, 2×CH₃).

2-Methylbutyl acetate. To a solution of 2-methyl-1-butanol (1.0 mL, 9.2 mmol) in 2.0 mL hexanes at RT was added 0.5 g of DMAP and stirred for 30 min. Then the solution was cooled to 0° C. and excess acetyl chloride (1.2 mL) was added and allowed to stirred at RT for 12 hrs. The reaction was quenched with water and extracted with hexanes (2×2 mL). The solvent was carefully removed under a stream of air. ¹H NMR (400 MHz, CDCl₃) δ 3.81 (dd, J=11 and 6 Hz, OCHₐCH_{b}CH), 3.72 (dd, J=11 and 6.8 Hz, OCHₐCH_{b}CH), 1.91 (s, CH₃CO), 1.56 (m, CHₐH_{b}CH₃), 1.32 (m, CH₃CH₂CH), 1.05 (m, CHₐH_{b}CH₃), 0.79 (d, J=7.2, CH₃CH₂), 0.77 (d, J=7.6 Hz, CH₃CH).

The other esters that were not commercially available were made following some of the acylation procedures set forth in Hoefle, G., et al. (1978).

Propanoic acid, 2-methyl,3-methylbutyl ester. Isobutyryl chloride (2 ml 19.1 mmol) was slowly added to a 0° C. solution of isoamyl alcohol (1 ml, 9.5 mmol), 4-dimethylaminopyridine (583 mg, 4.8 mmol), and pyridine (0.85 ml, 10.5 mmol) in dichloromethane. A precipitate was evident 5 minutes after addition was complete. After stirring 12 h under argon, the reaction was poured into 20 ml of 0.1 N HCl. The layers were separated and the aqueous layer was extracted with 20 ml of methylene chloride. The organic layers were combined and washed with 10 ml of saturated aqueous ammonium chloride then 10 ml of saturated aqueous sodium bicarbonate. The organic layers were dried over magnesium sulfate, filtered, and concentrated in vacuo. Purified by distillation through a 14 mm Vigreaux column (bp 60-62 C, 25 mm). The resulting clear, colorless oil was stirred over Amberlyst 15 to remove any remaining isobutyryl chloride. ¹H NMR (250 MHz, CDCl₃) 4.09 (t, 2H, J 6.7), 2.53 (m, 1H), 1.68 (m, 1H), 1.52 (q, 2H, J 6.5), 1.16 (d, 6H, J 7.0), 0.92 (d, 6H, J 6.5).

Propanoic acid, 2-methyl-ethyl ester. Isobutyryl chloride (2 ml 19.1 mmol) was slowly added to a 0° C. solution of ethyl alcohol (0.55 ml, 9.5 mmol), 4-dimethylaminopyridine (583 mg, 4.8 mmol), and pyridine (0.85 ml, 10.5 mmol) in dichloromethane. A precipitate was evident 5 minutes after addition was complete. After stirring 12 h under argon, the reaction was poured into 20 ml of 0.1 N HCl. The layers were separated and the aqueous layer was extracted with 20 ml of methylene chloride. The organic layers were combined and washed with 10 ml of saturated aqueous ammonium chloride then 10 ml of saturated aqueous sodium bicarbonate. The organic layers were dried over magnesium sulfate, filtered, and concentrated in vacuo. Purified by distillation through a 14 mm Vigreaux column (bp 102 C). ¹H (300 MHz, CDCl₃) 4.12 (q, 2H, J 7.2), 2.52 (m, 1H), 1.25 (t, 3H, J 6.9), 1.16(d, 6H, J 7.2).

1-Butanol, 3 methyl, acetate. Under an atmosphere of argon, acetyl chloride (6.5 ml, 91.8 mmol) was added dropwise to a 0° C. solution of isoamyl alcohol (5 ml, 45.9 mmol), N,N-dimethylpyridine (2.8 g, 23 mmol), and anhydrous pyridine (4.1 ml, 50.5 mol) in dichloromethane (92 ml). The reaction mixture was poured into 100 ml of 0.1 N HCl, and the resulting layers were separated. The organic layer was washed with 50 ml of saturated aqueous ammonium chloride then dried over magnesium sulfate. The organic layer was filtered and concentrated in vacuo to a clear oil. The resulting oil was purified by distillation (bp 134-136° C.) to give isoamyl acetate. ¹H NMR (300 MHz, CDCl₃) 4.08 (t, 2H, J 6.9), 2.03 (s, 3H), 1.68 (m, 1H), 1.51 (q, 2H, J 6.9), 0.92 (d, 6H, J 6.6).

### Example 10

### Synthetic Mixtures of Volatile Organic Compounds Isolatable from Muscodor

Several experiments show that artificial mixtures of volatile organic compounds provide activity against plant pathogenic fungi.

Activity of Synthetic Mixtures of Volatile Organic Compounds Isolatable from Muscodor albus Grown on Brown Rice Grits and Rye Grain

In one set of experiments, a Petri plate divided by plastic walls into 3 equal spaces was used for the assay. A plug of R. solani was placed in one section of the Petri plate containing PDA. On another section of the plate, a lx2 cm piece of sterile filter paper was loaded with the test compound(s). All plates were wrapped with two layers of saran film and incubated at room temperature. The head space in each plate was 65 ml. Removing the agar plug and placing it onto a fresh PDA Petri plate determined the viability of R. Solani exposed to each test compound. Control experiments were also conducted along side without test compound(s).

Isobutyric acid and 3-methyl-1-butanol exhibited a lethal effect on R. solani similar to that observed when R. solani is exposed to M. albus. (See Table 7.) In addition, a simple mixture of three compounds demonstrated such a lethal effect. This mixture contained 28.5 μl 2-methyl-1 butanol, 28.5 μl ethyl propionate and 3 μl isobutyric acid. (See Table 8.) Many other simple mixtures were tested (see Table 8) and gave inhibition of R. solani growth but were not lethal to the pathogen. In addition, mixtures containing six or seven volatile organic compounds, shown in Tables 9 and 10, demonstrated a lethal effect on R. solani. Therefore, it is possible to use artificial mixtures of volatile compounds to mimic the activity of M. albus.

Tables 7-10 set forth the results of the experiments described above. The diameter of R. solani colonies on untreated control plates was 70-72 mm. The (+) symbol in the viability column of each table above indicates continued viability of the organism after exposure to and removal from the given compound or mixture, while the (−) symbol indicates death of the organism. Multiple (+) and (−) symbols in the same column indicate the results of multiple trials.

TABLE 7
Effect of Each Volatile Organic Compound on R. solani

| | | | | Colony | |
|---|---|---|---|---|---|
| | | Amount | | diameter | |
| | | μl/65 mL | | at 3 days | Via- |
| Code | Compound | head space | ppm | (mm) | bility |
| A | 2-Methyl-1-butanol | (60 μl/65 mL) | 750 | no growth | (+) |
| | | 0.92 | | | |
| | 2-Methyl-1-butanol | (30 μl/65 mL) | 375 | 30.5 | (+) |
| | | 0.46 | | | |
| | 2-Methyl-1-butanol | (15 μl/65 mL) | 188 | 55 | (+) |
| | | 0.23 | | | |
| | 2-Methyl-1-butanol | (7 μl/65 mL) | 90 | 66 | (+) |
| | | 0.11 | | | |
| B | 2-Methylbutyl acetate | 0.92 | 940 | 41 | (+) |
| | 2-Methylbutyl acetate | 0.46 | 470 | 56 | (+) |
| | 2-Methylbutyl acetate | 0.23 | 235 | 70 | (+) |
| | 2-Methylbutyl acetate | 0.11 | 112 | 70 | (+) |
| C | Isobutyl alcohol | 0.92 | 737 | 15 | (+) |
| | Isobutyl alcohol | 0.46 | 369 | 58 | (+) |
| | Isobutyl alcohol | 0.23 | 184 | 70 | (+) |
| | Isobutyl alcohol | 0.11 | 90 | 70 | (+) |
| D | Methyl 2- | 0.92 | 814 | 23.3 | (+) |
| | methylbutyrate | | | | |
| E | Methyl isobutyrate | 0.46 | 820 | 32 | (+) |
| | Methyl isobutyrate | 0.23 | 410 | 45.5 | (+) |
| | Methyl isobutyrate | 0.11 | 196 | 56 | (+) |
| F | Ethyl propionate | 0.92 | 819 | 13.5 | (+) |
| G | Ethyl isobutyrate | 0.92 | 798 | 36.5 | (+) |
| H | Ethyl butyrate | 0.92 | 809 | no growth | (+) |
| I | Phenethyl alcohol | 0.46 | 940 | 67.5 | |
| | Phenethyl alcohol | 0.23 | 470 | 66.5 | |
| J | Isobutyric acid | 0.92 | 873 | no growth | no |
| | Isobutyric acid | 0.46 | 437 | 4 | (+/−) |
| | Isobutyric acid | 0.23 | 218 | 12 | (+) |
| K | 3-methyl-1-butanol | 0.92 | 744 | no growth | no |
| | 3-methyl-1-butanol | 0.46 | 372 | fuzz | (+) |
| | 3-methyl-1-butanol | 0.23 | 186 | 70 | |
| | 3-methyl-1-butanol | 0.11 | 89 | 70 | |

TABLE 8
Effect of Mixtures of Volatile Organic Compounds

| | | | Colony | |
|---|---|---|---|---|
| | | | Diameter | |
| | | | at 3 | |
| Mixture | Code | Amount/65 mL head space) | days (mm) | Viability |
| 1 | A/C | 30 μl each (0.92) | 7.5 | (+) |
| 2 | A/F | 30 μl each (0.92) | no growth/ | (+) |
| | | | fuzz | |
| 3 | A/H | 30 μl each (0.92) | no growth | (+) |
| 4 | C/H | 30 μl each (0.92) | 12.5 | (+) |
| 5 | F/H | 30 μl each (0.92) | 11 | (+) |
| 6 | C/F | 30 μl each (0.92) | 14.5 | (+) |
| 7 | A/C/H | 20 μl each (0.92) | no growth/ | (+) |
| | | | fuzz | |
| 8 | A/F/H | 20 μl each (0.92) | 5 | (+) |
| 9 | A/C/F/H | 15 μl each (0.92) | fuzz | (+) |
| 10 | C/F/H | 20 μl each (0.92) | 18.5 | (+) |
| 11 | A/C/J | 28.5 μl A/C + 3 μl J | no growth | (+) |
| 12 | A/F/J | 28.5 μl A/F + 3 μl J | no growth | no |
| 13 | A/H/J | 28.5 μl A/H + 3 μl J | no growth | (+/−) |
| 14 | C/H/J | 28.5 μl C/H + 3 μl J | fuzz/9.0 | (+) |
| 15 | F/H/J | 28.5 μl F/H + 3 μl J | 6/ | (+) |
| | | | no growth | |
| 16 | C/F/J | 28.5 μl C/F + 3 μl J | no growth/ | (+) |
| | | | fuzz | |
| 17 | F/J/K | 28.5 μl F/K + 3 μl J | no growth | (+) |
| 18 | H/J/K | 28.5 μl H/K + 3 μl J | no growth | (+) |
| 19 | A/C/H/J | 19 μl A/C/H + 3 μl J | no growth | (+/−) |
| 20 | A/F/H/J | 19 μl A/F/H + 3 μl J | no growth | (+) |
| 21 | C/F/H/J | 19 μl C/F/H + 3 μl J | no growth | (+) |
| 22 | A/C/F/H/J | 14.25 μl A/C/F/H + 3 μl J | no growth | (+) |
| 23 | K/C/F/H/J | 14.25 μl K/C/F/H + 3 μl J | no growth | (+) |
| | | Control | 70-72 | |

TABLE 9
Effect of Various Concentrations of a Mixture
of Several Volatile Organic Compounds on R. solani
Combination (amount/65 mL head space)

| | Mixture→ | | | | | | |
|---|---|---|---|---|---|---|---|
| Compound | A (μl) | B (μl) | 1.25x B | 1.5x B | 1.75x B | C (μl) | D (μl) |
| 2-Methyl-1-butanol | 16 | 32 | 40 | 48 | 56 | 64 | 96 |
| Ethyl butyrate | 7 | 14 | 17.5 | 21 | 24.5 | 28 | 42 |
| Isobutyl alcohol | 1.5 | 3 | 3.75 | 4.5 | 5.25 | 6 | 9 |
| Phenethyl alcohol | 1.5 | 3 | 3.75 | 4.5 | 5.25 | 6 | 9 |
| Ethyl isobutyrate | 1 | 2 | 2.5 | 3 | 3.5 | 4 | 6 |
| 2-Methylbutyl | 2 | 4 | 0 | 0 | 0 | 8 | 12 |
| acetate | | | | | | | |
| Isobutyric acid | 1.5 | 3 | 3.75 | 4.5 | 5.25 | 6 | 9 |
| Colony growth | 10.3 | 3.3 | no | no | no growth | no | no |
| (mm) | | | growth | growth | | growth | growth |
| Viability | (+) | (+/−) | (+/−/−) | (+/+/−) | (−/−/−) | (−) | (−) |

TABLE 10
Effect of a Mixture of Several Volatile
Organic Compounds on R. solani
Amount/65 mL head space

| Compound | A (μl) | B (μl) |
|---|---|---|
| 3-Methyl-1-butanol | 16 | 56 |
| Ethyl butyrate | 7 | 24.5 |
| Isobutyl alcohol | 1.5 | 5.25 |
| Phenethyl alcohol | 1.5 | 5.25 |
| Ethyl isobutyrate | 1 | 3.5 |
| Isobutyric acid | 1.5 | 5.25 |
| Colony growth (mm) | no growth/fuzz | no growth |
| Viability | (+) | (−) |

Activity of Synthetic Mixtures of Volatile Organic Compounds Isolatable from Muscodor albus Grown on Potato Dextrose Agar

In another set of experiments, test solutions were prepared by placing the volatile organic compounds isolatable from Muscodor albus cultures grown on potato dextrose agar (PDA) in vials in the relative proportions that they occurred in the gas phase of such cultures. The test mixture was placed in a presterilized microcup (4×6 mm) located in the center of a Petri plate containing PDA. When not in use, the mixture was stored at 0° C. The test organisms, freshly growing and excised on 3 mm³ agar blocks (at least 3 agar blocks per test fungus), were placed 2-3 cm from the microcup and the plate wrapped with two layers of parafilm and grown for 2 or more days at 23° C. Measurements were made on mycelial growth from the edge of the agar blocks. However, in the case of bacteria and Candida albicans they were streaked on the test side of the PDA plate and checked for new visible growth and viability by restreaking from the original area of the agar plate that had been inoculated. Appropriate controls were also set up in which no test solution was placed into the microcup. Tests on 3.2-90 μl of the artificial mixture per 50 CC of air space above the PDA plate were done on 3 replicates in order to obtain IC₅₀ data for each test organism. Viability of the test microbes was made by aseptically removing the small agar block and placing it on a PDA plate and observing growth after 1-3 days.

As shown in Table 11, the growth of all of the pathogens exposed to the synthetic mixture of the Muscodor volatiles isolatable from Muscodor albus grown on PDA was inhibited, and the majority of the pathogens were killed by exposure to the synthetic mixture.

In Table 11, below, the amount of each positively identified compound used in the artificial mixture was obtained by applying the electron ionization cross section (% of the total area) of the compound obtained in the GC/MS analysis. (See Table 6.) The compounds in Table 6 preceded by the symbol # were not included in the synthetic mixture. The symbol # in. Table 11 below means the data for a particular organism was not measured in this experimental design.

TABLE 11
The effects of the volatile compounds of M. albus and a
synthetic mixture of M. albus compounds on a group of test microbes

| | % Growth | Viability | | | |
|---|---|---|---|---|---|
| | over control | after | IC₅₀ in | % Growth | Viability |
| | after a 2 | 3 days | artificial | (mm) over | after 3 days |
| | day | exposure | atmosphere | control in | exposure |
| | exposure to | to M. albus | for 2 days | artificial | artificial |
| Test Microbe | M. albus | culture | (μl/CC) | atmosphere | atmosphere |
| Pythium ultimum | 0 | Dead | 0.48 ± 0.01 | 0 | Dead |
| Phytophthora | 0 | Dead | 0.29 ± 0.06 | 0 | Dead |
| cinnamoni | | | | | |
| Penicillium | 0 | Dead | # | # | # |
| expansum | | | | | |
| Rhizoctonia solani | 0 | Dead | 0.08 ± 0.02 | 0 | Dead |
| Ustilago hordei | 0 | Dead | 0.31 ± 0.09 | 0 | Dead |
| Stagnospora | 0 | Dead | 0.15 ± 0 | 0 | Dead |
| nodorum | | | | | |
| Sclerotinia | 0 | Dead | 0.17 ± 0.05 | 0 | Alive |
| sclerotiorum | | | | | |
| Scerotinia minor | 0 | Dead | # | # | # |
| Aspergillus | 0 | Dead | 0.41 ± 0.05 | 0 | Alive |
| fumigatus | | | | | |
| Monilinia fructicola | 0 | Dead | # | # | # |
| Fusarium solani | 19.4 ± 0.284 | Alive | 1.13 ± 0.07 | | |
| Fusarium oxysporum | 4 | Alive | # | # | # |
| Verticillum dahliae | 0 | Dead | 0.3 ± 0 | 0 | Dead |
| Cercospora beticola | 17.5 ± 3.5 | Alive | 0.12 ± 0.15 | 8 ± 2 | Alive |
| Tapesia yallundae | 0 | Dead | 0.64 ± 0 | 0 | Dead |
| Xylaria sp. | 25 ± 0 | Alive | 0.41 ± 0.03 | 0 | Alive |
| Muscodor albus | 100 ± 0 | Alive | 0.6 ± 0 | 17.5 ± 7.5 | Alive |
| Escherichia coli | 0 | Dead | # | 0 | Dead |
| Staphlococcus | 0 | Dead | # | 0 | Dead |
| aureus | | | | | |
| Micrococcus luteus | 0 | Dead | # | 0 | Dead |
| Candida albicans | 0 | Dead | # | Trace | Alive |
| Bacillus subtilus | 0 | Alive | # | 0 | Alive |

To determine the relative biological activity of each class of compounds, individual classes were also tested in the relative amounts in which they occur at the optimum concentration of the entire mixture, which is 60 μl of test mixture per 50 CC of air space above the culture in a standard Petri plate. For instance, the esters represent 44% of the mixture of the identified volatiles and were tested at 26.4 μl/50 CC (0.53 μl/CC) air space and the same procedure was used for each of the other classes of compounds that were identified. This was done with a selected group of 7 test fungi. Each group of compounds possessed some inhibitory activity against the test organisms (Table 12). However, on a comparative basis the esters had more inhibitory activity than any other group of compounds (Table 12).

Each compound in the class of esters was individually evaluated. When a comparable test on each ester was conducted as per the conditions in Table 6,1-butanol, 3-methyl, acetate (3-methylbutyl acetate), almost completely mimicked the results of all esters shown in Table 6. It represented 62% of all of the identified combined esters and was therefore tested at the level of 0.32 μl/CC. Additionally, minimal inhibitory bioactivity was displayed by propionic acid, 2-methyl, 3-methylbutyl ester and little or no activity was noted on the part of the other esters. Although the esters, and the 1-butanol, 3 methyl-acetate had inhibitory activity in the bioassay tests, under no conditions in any test, was death of any test fungus observed under the standard 3 day exposure period (Table 12). This is a significant observation, since the death of test organisms was noted in both the complete artificial atmosphere and in the natural Petri plate atmosphere of M. albus. The result strongly suggests that an additive or synergistic mechanism is operational in the case of the M. albus volatiles. Thus, while each class of compounds possesses more or less inhibitory activity, a mixture of the ingredients is needed to bring about death of the test fungi and bacterium (Table 11). All measurements of mycelial growth compared to the untreated control were made as described above.

TABLE 12
The inhibitory influence of each class
of volatile compounds is expressed as the % of the
test microbe growth as compared to a control
not in the presence of the test compounds

| | Alcohols | Esters | Ketones | Acids | Lipids |
|---|---|---|---|---|---|
| | 0.48 μl/cc | 0.53 μl/cc | 0.02 μl/cc | 0.09 μl/cc | 0.08 μl/cc |
| | % growth | % growth | % growth | % growth | % growth |
| Test Microbe# | of control | of control | of control | of control | of control |
| Phythium Ultimum | 11.2 ± 4 | 0 | 67.5 ± 7 | 40.9 ± 3 | 75 ± 0 |
| Rhizoctonia solani | 55 ± 5 | 0 | 67.5 ± 7.5 | 67.5 ± 7.5 | 40 ± 0 |
| Tapesia yallundae | 35 ± 15 | 0 | 75 ± 25 | 100 ± 0 | 100 ± 0 |
| Xylaria sp. | 75 ± 25 | 0 | 100 ± 0 | 100 ± 0 | 100 ± 0 |
| Sclerotinia sclerotiorum | 29 ± 3 | 8.1 ± 1.5 | 20.6 ± 12 | 40 ± 0 | 78 ± 2 |
| Cercospora beticola | 58 ± 8 | 5 ± 5 | 100 ± 0 | 83 ± 17 | 100 ± 0 |
| Fusarium solani | 70 ± 10 | 55 ± 5 | 90 ± 10 | 80 ± 20 | 80 ± 10 |

Synergism between Volatile Organic Compounds

Synergism between the volatile organic components produced by M. albus was studied using the method of Limpel as described by Richer (Richer, D. L. 1987). The determination of synergy as described by Limpel can be represented by the following equation:

Eₑ=X+Y−(XY/100)

Where Eₑ is the expected additive effect of the two antifungal compounds, X is the observed percentage of inhibition of the test organism when antifungal agent A is applied alone at the rate used in the mixture, and Y is the observed percentage inhibition of the test organism when antifungal agent B is used alone at the rate used in the mixture. If the observed effect (Eₒ) is greater than the expected effect then synergism is said to have been exhibited.

Experimental studies were set up to determine if synergy exists between the components produced by M. albus. The biological activity of individual volatile organic compounds and mixtures of volatile organic compounds was tested according to the method described in Example 10 above. Results are shown in Table 13. The observed inhibitory effects of the volatile organic compounds were then compared to the expected additive effects as calculated from the equation above. This comparison is also shown in Table 13. The results demonstrate that mixtures of volatile organic compounds produced by M. albus provide synergistic antifungal activity where the observed inhibitory effect is greater than the expected effect.

The codes in the table below correspond to the code for each volatile organic compound set forth in Table 7, above.

TABLE 13
Effect of Mixtures of Volatile Organic Compounds

| | | | | Expected Additive | |
|---|---|---|---|---|---|
| | | | Percent Inibition of | Percent Inhibition | |
| | | Amount/65 mL head | Colony growth at 3 | of Colony Growth | |
| Mixture | Code | space | days | at 3 Days | Viability |
| 1 | A | 30 μl (0.92) | 68 | N/A | (+) |
| 2 | C | 30 μl (0.92) | 0 | N/A | (+) |
| 3 | F | 30 μl (0.92) | 76 | N/A | (+) |
| 4 | H | 30 μl (0.92) | 86 | N/A | (+) |
| 5 | A/C | 30 μl each (0.92) | 86 | 68 | (+) |
| 6 | A/F | 30 μl each (0.92) | 100 | 92 | (+) |
| 7 | A/H | 30 μl each (0.92) | 100 | 92 | (+) |
| 8 | C/F | 30 μl each (0.92) | 83 | 76 | (+) |
| 9 | Control | No compound | 0 | 0 | (+) |

### REFERENCES

1. Heathcock, R. and Ratcliff, R. (1971) J. Am. Chem. Soc. 93: 1746.

2. Hoefle, G., et al., (1978) Vorbrueggen, Agnew. Chem., Int. Ed. Engl. 17: 569.

3. Lin, J., et al. (1991) Biotechnology and Bioengineering 38: 273-279.

4. Nelson, P. V. (1998) Greenhouse Operation and Management 5h ed. Prentice-Hall.

5. Richer, D. L. (1987) Pestic Sci. 19: 309-319.

6. Strobel, G. A., et al. (2001) Microbiology 147: 2943-2950.

7. Strobel, G. A., et al. (1996) Microbiology 142: 435-440.

8. Strobel, G. A., et al. (2000) Mycotaxon. 76: 257-266.

9. Worapong, J., et al. (2001) Mycotaxon. 79: 67-69.

1. A Muscodor carrier formulation comprising:
a carrier,
a stabilizing agent, and
a culture of Muscodor albus,
wherein the culture and the stabilizing agent are adhered to the carrier.

2. The Muscodor carrier formulation of claim 1 wherein the carrier is grain.

3. The Muscodor carrier formulation of claim 2 wherein the grain is selected from the group consisting of corn, rye, barley, rice, wheat, oat bean, and soy.

4. The Muscodor carrier formulation of claim 1 wherein the carrier is absorptive material containing nitrogen and carbon sources.

5. The Muscodor carrier formulation of claim 1 wherein the stabilizing agent is a carbohydrate.

6. The Muscodor carrier formulation of claim 5 wherein the carbohydrate is selected from the group consisting of lactose, sucrose and trehalose.

7. The Muscodor carrier formulation of claim 5 wherein the carbohydrate is lactose.

8. The Muscodor carrier formulation of claim 1 further comprising a matrix wherein the carrier, culture, and stabilizing agent are encapsulated by the matrix.

9. The Muscodor carrier formulation of claim 8 wherein the matrix is a hydrogel.

10. A method for preparing a Muscodor carrier formulation comprising:
growing a culture of Muscodor;
inoculating a carrier with the culture of Muscodor;
adding a stabilizing agent to the carrier; and
drying the carrier.

11. The method of claim 10 wherein the stabilizing agent is a carbohydrate.

12. The method of claim 11 wherein the carbohydrate is selected from the group comprising sucrose, trehalose, and lactose.

13. The method of claim 12 wherein the carbohydrate is lactose.

14. The method of claim 10 wherein the carrier is a grain.

15. The method of claim 14 wherein the grain is selected from the group consisting of corn, rye, barley, rice, wheat, oat bean, and soy.

16. The method of claim 10 further comprising encapsulating the carrier before drying the carrier.

17. A synthetic mixture comprising pesticidally effective amounts of at least two of the following volatile organic compounds isolatable from an isolated culture of Muscodor albus: 2-methyl-1-butanol, isobutyl alcohol, isobutyric acid, 3-methyl-1-butanol, 3-methylbutyl acetate, and ethyl propionate.

18. The mixture of claim 17 wherein the at least two volatile organic compounds comprise isobutyric acid and at least one of 2-methyl-1-butanol, isobutyl alcohol, ethyl propionate, and 3-methyl-1-butanol.

19. A synthetic mixture comprising pesticidally effective amounts of at least two of the following volatile organic compounds isolatable from an isolated culture of Muscodor albus: 2-methyl-1-butanol, isobutyl alcohol, methyl isobutyrate, isobutyric acid, 3-methyl-1-butanol, 3-methylbutyl acetate, and ethyl butyrate.

20. The mixture of claim 19 wherein the at least two volatile organic compounds comprise isobutyric acid and at least one of 2-methyl-1-butanol, isobutyl alcohol, ethyl butyrate, and 3-methyl-1-butanol.

21. A synthetic mixture comprising pesticidally effective amounts of at least two volatile organic compounds isolatable from an isolated culture of Muscodor albus grown on potato dextrose agar.

22. The mixture of claim 21 wherein the at least two volatile organic compounds comprise 3-methylbutyl acetate and propionic acid, 2-methyl, 3-methylbutyl ester.

23. A synthetic mixture comprising pesticidally effective amounts of at least two volatile organic compounds isolatable from an isolated culture of Muscodor albus grown on brown rice grit.

24. A synthetic mixture comprising pesticidally effective amounts of at least three volatile organic compounds isolatable from at least one of an isolated culture of Muscodor albus grown on rye grain, an isolated culture of Muscodor albus grown on brown rice grit, and an isolated culture of Muscodor albus grown on potato dextrose agar.

25. The mixture of claim 24 wherein the at least three volatile organic compounds comprise isobutyric acid and at least two of 2-methyl-1-butanol, isobutyl alcohol, ethyl propionate, ethyl butyrate, and 3-methyl-1-butanol.

26. The mixture of claim 24 wherein the at least three volatile organic compounds comprise 2-methyl-1-butanol and at least two of isobutyl alcohol, ethyl propionate, and ethyl butyrate.

27. The mixture of claim 24 wherein the at least three volatile organic compounds comprise ethyl butyrate, isobutyl alcohol, and ethyl propionate.

28. The mixture of claim 24 wherein the isolated culture of Muscodor albus is grown on rye grain.

29. The mixture of claim 24 wherein the at least three volatile organic compounds comprise 2-methyl-1-butanol, ethyl butyrate, isobutyl alcohol, phenethyl alcohol, ethyl isobutyrate, 2-methylbutyl acetate, and isobutyric acid.

30. The mixture of claim 24 wherein the at least three volatile organic compounds comprise 3-methyl-1-butanol, ethyl butyrate, isobutyl alcohol, phenethyl alcohol, ethyl isobutyrate, and isobutyric acid.

31. A method for inhibiting the growth of organisms selected from the group consisting of microbes, insects, and nematodes comprising exposing the organism or a habitat of the organism to an effective amount of the Muscodor carrier formulation of claim 1.

32. A method for inhibiting the growth of organisms selected from the group consisting of microbes, insects, and nematodes comprising exposing the organism or a habitat of the organism to the synthetic mixture of claim 17.

33. A method for inhibiting the growth of organisms selected from the group consisting of microbes, insects, and nematodes comprising exposing the organism or a habitat of the organism to the synthetic mixture of claim 19.

34. A method for inhibiting the growth of organisms selected from the group consisting of microbes, insects, and nematodes comprising exposing the organism or a habitat of the organism to the synthetic mixture of claim 24.

35. A method for inhibiting the growth of organisms selected from the group consisting of microbes, insects, and nematodes comprising exposing the organism or a habitat of the organism to an effective amount of 3-methylbutyl acetate.

36. A method for inhibiting the growth of organisms selected from the group consisting of microbes, insects, and nematodes comprising exposing the organism or a habitat of the organism to an effective amount of 3-methyl-1-butanol.

37. A method for inhibiting the growth of organisms selected from the group consisting of microbes, insects, and nematodes comprising exposing the organism or a habitat of the organism to an effective amount of isobutyl alcohol.

38. A method for inhibiting the growth of organisms selected from the group consisting of microbes, insects, and nematodes comprising exposing the organism or a habitat of the organism to an effective amount of methyl 2-methylbutyrate.

39. A method for inhibiting the growth of organisms selected from the group consisting of microbes, insects, and nematodes comprising exposing the organism or a habitat of the organism to an effective amount of methyl isobutyrate.

40. A method for inhibiting the growth of organisms selected from the group consisting of microbes, insects, and nematodes comprising exposing the organism or a habitat of the organism to less than 2500 ppm 2-methyl-1-butanol.

41. A method for inhibiting the growth of organisms selected from the group consisting of microbes, insects, and nematodes comprising exposing the organism or a habitat of the organism to less than 2800 ppm isobutyric acid.
